(19) United States
(12) Patent Application Publication
(10) Pub. No.: US 2023/0165652 A1
(43) Pub. Date: Jun. 1, 2023

(54) HAND DETECTION FOR ROBOTIC SURGICAL SYSTEMS

(71) Applicant: Covidien LP, Mansfield, MA (US)

(72) Inventors: Steven J. Levine, Framingham, MA (US); Albert Dvornik, Somerville, MA (US); William J. Peine, Ashland, MA (US); Mantena VR. Raju, Medford, MA (US); Chen Chen, Malden, MA (US)

(21) Appl. No.: 17/916,668

(22) PCT Filed: Mar. 3, 2021
(86) PCT No.: PCT/US21/20569

§ 371 Date: Oct. 3, 2022

Related U.S. Application Data
(60) Provisional application No. 63/013,018, filed on Apr. 21, 2020.

(51) Int. Cl. A61B 34/00; A61B 34/37

(52) U.S. Cl. CPC A61B 34/74; A61B 34/37; A61B 34/25

(57) ABSTRACT

A robotic surgical system includes a robot system, a user interface, a hand detection system, and a processing unit. The robot system includes a tool coupled to an arm. The user interface includes a handle assembly including a body portion having a proximal end portion, and a first actuator movable between open and closed positions. The hand detection system includes a first sensor disposed within the first actuator for detecting finger presence on the first actuator, a second sensor disposed on the proximal end portion for detecting palm presence about the proximal end portion, and an encoder disposed within the body portion for detecting position of the first actuator relative to the body portion. The processing unit is electrically coupled to the first, second, and third sensors for receiving and processing data from the first, second, and third sensors.

### CROSS-REFERENCE TO RELATED APPLICATIONS

This application is a 371 National Stage Application of International Application No. PCT/US2021/020569, filed Mar. 3, 2021, which claims benefit of U.S. Provisional Patent Application No. 63/013,018, filed Apr. 21, 2020, the entire contents of each of which is hereby incorporated herein by reference.

### FIELD

The present disclosure is generally related to handle assemblies of a user interface of a robotic surgical system that allows a clinician to control a robot system including a robotic surgical instrument of the robotic surgical system during a surgical procedure.

### BACKGROUND

Robotic surgical systems have been used in minimally invasive medical procedures. During such medical procedures, a robotic surgical system is controlled by a surgeon interfacing with a user interface. The user interface allows the surgeon to manipulate an end effector of a robot system that acts on a patient. The user interface includes control arm assemblies that are moveable by the surgeon to control the robot system.

Hand detection is a safety feature for a robotic surgical system. Without hand detection, there could be unintended motion of the robot system while in the patient (e.g., the control arm assemblies drift or are accidently knocked) if the surgeon removes his or her hands from handle assemblies of the control arm assemblies.

### SUMMARY

The techniques of the present disclosure generally relate to robotic surgical systems including a hand detection system for detecting the presence or absence of the hands of a clinician on handle assemblies of the robotic surgical system. The robotic surgical systems can lock movement of one or more arms and/or tools of a robot system when no hand is present on one or more of the handle assemblies. This minimizes unintended robot system motion if a handle assembly drifts or is accidentally moved when not being held by a clinician to improve safety.

The hand detection system utilizes a plurality of sensors in the handle assemblies. The data from the plurality of sensors are fused together so that the final output of the hand detection system is robust to noise as compared to hand detection systems utilizing a single sensor. The hand detection system integrates data from multiple sources (e.g., the plurality of sensors) to produce more consistent, accurate, and useful information than that provided by a single data source (e.g., a single sensor).

In one aspect, the present disclosure provides a robotic surgical system including a robot system, a user interface, a hand detection system, and a processing unit. The robot system includes an arm and a tool coupled to the arm. The user interface includes a handle assembly including a body portion having a proximal end portion and a distal end portion. The body portion includes a first actuator movable between an open position and a closed position. The hand detection system includes a first sensor disposed within the first actuator of the handle assembly for detecting finger presence on the first actuator, a second sensor disposed on the proximal end portion of the handle assembly for detecting palm presence about the proximal end portion, and an encoder disposed within the body portion of the handle assembly for detecting position of the first actuator relative to the body portion. The processing unit is electrically coupled to the first, second, and third sensors for receiving and processing data from the first, second, and third sensors.

The first sensor may be a capacitive sensor, the second sensor may be an infrared sensor, and/or the third sensor may be an encoder.

The hand detection system may have an initialization state in which the hand detection system utilizes data from only the first and third sensors, and/or an operation stage in which the hand detection system utilizes data from the first, second, and third sensors. When in the initialization state, the first actuator may move through a full range of motion between the open and closed positions. The first sensor may detect a capacitance value at each of a plurality of points through the full range of motion and the third sensor may generate an encoder count at each of the plurality of points.

The hand detection system may include a lookup table including a baseline curve of the capacitance values as a function of the encoder counts and a calibrated curve of threshold capacitance values as a function of the encoder counts. When in the operation stage, the first sensor may detect a real-time capacitance value and the third sensor may detect a real-time encoder count. The real-time capacitance value and the real-time encoder count may be compared to the lookup table to identify a positive or negative finger presence state of the handle assembly.

When the hand detection system is in the operation stage, the second sensor may detect a real-time value which is compared to a threshold value to identify a positive or negative palm presence state of the handle assembly.

The tool of the robot system may be a jaw assembly including opposed jaw members. When the first actuator is in the open position, the jaw members may be in an open configuration, and when the first actuator is in the closed position, the jaw members may be in a closed configuration.

In another aspect, the present disclosure provides a method of detecting hand presence on a handle assembly of a robotic surgical system including: initializing a hand detection system of a robotic surgical system by: sweeping a first actuator of a handle assembly of the robotic surgical system through a full range of motion from an open position to a closed position; recording capacitive values obtained from a first sensor disposed within the first actuator of the handle assembly and encoder counts obtained from a third sensor disposed within a body portion of the handle assembly at a plurality of points through the full range of motion; and constructing a lookup table with the capacitive values as a function of encoder counts at the plurality of points; and operating the hand detection system by: comparing a real-time capacitive value of the first sensor and a real-time encoder count of the third sensor against the lookup table to identify a positive or negative finger presence state of the handle assembly.

Operating the hand detection system may further include comparing a real-time value of a second sensor disposed in a proximal end portion of the handle assembly against a threshold value to identify a positive or negative palm presence state of the handle assembly.

Constructing the lookup table may further include generating a baseline curve of the capacitance values as a function of the encoder counts and a calibrated curve of threshold capacitance values as a function of the encoder counts.

Comparing the real-time capacitive value of the first sensor and the real-time encoder count of the third sensor against the lookup table may further include determining if the real-time capacitive value exceeds the threshold capacitance value.

The method may further include identifying a hand presence detection state where, if positive finger and palm presence states are identified by the hand detection system, a positive hand presence state is identified and movement of the handle assembly results in a corresponding movement of a tool of a robot system, and if negative finger and palm presence states are identified by the hand detection system, a negative hand presence state prevents movement of the tool of the robot system in response to movement of the handle assembly.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic illustration of a robotic surgical system including a robot system and a user interface, in accordance with an embodiment of the present disclosure;

FIG. 2 is an enlarged perspective view of control arm assemblies of the user interface of FIG. 1;

FIG. 3 is a perspective view of a handle assembly of one of the control arm assemblies of FIG. 2, with a hand of a clinician shown in phantom;

FIG. 4 is a perspective view of a tool of the robot system of FIG. 1;

FIG. 5 is a top, perspective view, with parts removed, of the handle assembly of FIG. 3;

FIGS. 6 and 7 are graphs showing capacitance values as a function of encoder counts for handle assemblies of the robotic surgical system of FIG. 1, in accordance with an example of the present disclosure; and

FIG. 8 is a lookup table showing capacitance values as a function of encoder counts, in accordance with an example of the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor (e.g., a surgeon), nurse, or any other care provider and may include support personnel. The term “patient” refers to a human or other animal. Throughout this description, the term “proximal” refers to a portion of a system, device, or component thereof that is closer to a hand of a clinician, and the term “distal” refers to a portion of the system, device, or component thereof that is farther from the hand of the clinician.

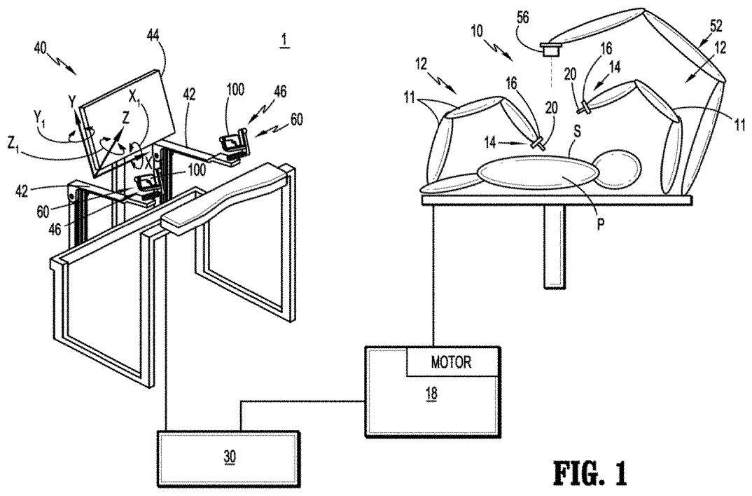

Turning now to FIG. 1, a robotic surgical system 1 in accordance with the present disclosure is shown. The robotic surgical system 1 includes a robot system 10, a processing unit 30, and an operating console or user interface 40. The robot system 10 generally includes linkages 11 and a robot base 18. The linkages 11 moveably support an end effector, robotic surgical instrument, or tool 20 which is configured to act on tissue of a patient “P” at a surgical site “S.” The linkages 11 may form arms 12, with each arm 12 having an end 14 that supports the tool 20. In addition, the ends 14 of each of the arms 12 may include an imaging device 16 for imaging the surgical site “S,” and/or a tool detection system (not shown) that identifies the tool 20 (e.g., a type of surgical instrument) supported or attached to the end 14 of the arm 12.

The processing unit 30 electrically interconnects the robot system 10 and the user interface 40 to process and/or send signals transmitted and/or received between the user interface 40 and the robot system 10, as described in further detail below.

The user interface 40 includes a display device 44 which is configured to display three-dimensional images. The display device 44 displays three-dimensional images of the surgical site “S” which may include data captured by the imaging devices 16 positioned on the ends 14 of the arms 12 and/or include data captured by imaging devices that are positioned about the surgical theater (e.g., an imaging device positioned within the surgical site “S,” an imaging device positioned adjacent the patient “P”, an imaging device 56 positioned at a distal end of an imaging arm 52). The imaging devices (e.g., imaging devices 16, 56) may capture visual images, infra-red images, ultrasound images, X-ray images, thermal images, and/or any other known real-time images of the surgical site “S.” The imaging devices 16, 56 transmit captured imaging data to the processing unit 30 which creates three-dimensional images of the surgical site “S” in real-time from the imaging data and transmits the three-dimensional images to the display device 44 for display.

The user interface 40 includes control arms 42 which support control arm assemblies 46 to allow a clinician to manipulate the robot system 10 (e.g., move the arms 12, the ends 14 of the arms 12, and/or the tools 20). The control arm assemblies 46 are in communication with the processing unit 30 to transmit control signals thereto and to receive feedback signals therefrom which, in turn, transmit control signals to, and receive feedback signals from, the robot system 10 to execute a desired movement of robot system 10.

Each control arm assembly 46 includes a gimbal 60 operably coupled to the control arm 42 and an input device or handle assembly 100 operably coupled to the gimbal 60. Each of the handle assemblies 100 is moveable through a predefined workspace within a coordinate system having “X,” “Y,” and “Z” axes to move the ends 14 of the arms 12 within a surgical site “S.” As the handle assemblies 100 are moved, the tools 20 are moved within the surgical site “S.” It should be understood that movement of the tools 20 may also include movement of the arms 12 and/or the ends 14 of the arms 12 which support the tools 20.

The three-dimensional images on the display device 44 are orientated such that the movement of the gimbals 60, as a result of the movement of the handle assemblies 100, moves the ends 14 of the arms 12 as viewed on the display device 44. It will be appreciated that the orientation of the three-dimensional images on the display device 44 may be mirrored or rotated relative to a view from above the patient “P.” In addition, it will be appreciated that the size of the three-dimensional images on the display device 44 may be scaled to be larger or smaller than the actual structures of the surgical site “S” to permit a clinician to have a better view of structures within the surgical site “S.” For a detailed discussion of scaling of handle assembly movement, reference may be made to commonly owned International Patent Application Serial No. PCT/US16/65588.

For a detailed discussion of the construction and operation of a robotic surgical system, reference may be made to U.S. Pat. No. 8,828,023.

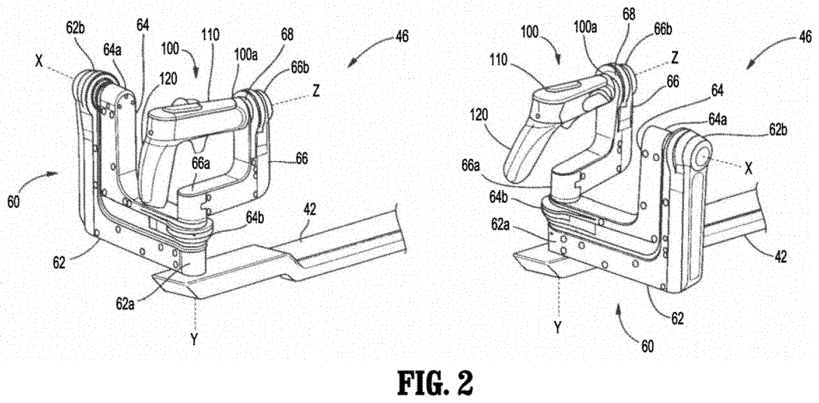

Referring now to FIG. 2, each gimbal 60 of the control arm assemblies 46 includes an outer link 62, an intermediate link 64, and an inner link 66. The outer link 62 includes a first end 62a pivotably connected to the control arm 42 and a second end 62b pivotably connected to a first end 64a of the intermediate link 64 such that the intermediate link 64 is rotatable, as indicated by arrow “Xi” (FIG. 1), about the “X” axis. The intermediate link 64 includes a second end 64b pivotably connected to a first end 66a of the inner link 66 such that the inner link 66 is rotatable, as indicated by arrow “Yi” (FIG. 1), about the “Y” axis. The inner link 66 includes a second end 66b having a connector 68 configured to releasably engage a distal end portion 100a of the handle assembly 100 such that the handle assembly 100 is rotatable, as indicated by arrow “Z₁” (FIG. 1), about the “Z” axis.

In embodiments, the outer, intermediate, and inner links 62, 64, 66 are each substantially L-shaped frames that are configured to nest within each other. However, it should be understood that the outer, intermediate, and inner links 62, 64, 66 may be any shape so long as the “X,” “Y,” and “Z” axes are orthogonal to each other in the zero or home position (see e.g., FIG. 2). It should also be understood that other gimbal configurations may be utilized in the control arm assemblies 46 so long as the movement of the handle assemblies 100 about the “X,” “Y,” and “Z” axes is maintained. Further still, the connector 68 of the gimbal 60 may allow for different sized or kinds of handle assemblies 100 to be used to control the arms 12 and/or the tools 20 of the robot system 10.

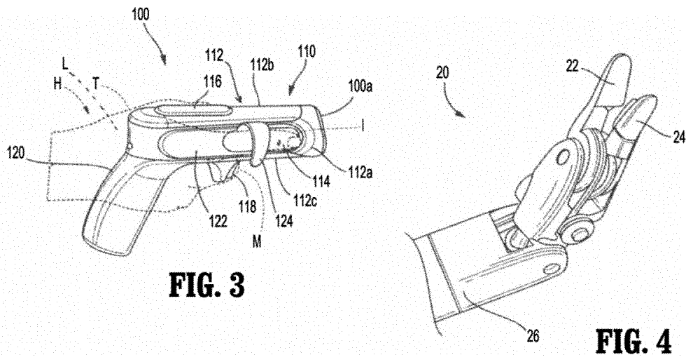

As shown in FIGS. 2 and 3, the handle assembly 100 of each of the control arm assemblies 46 includes a body portion 110 and a grip portion 120. The body portion 110 includes a housing 112 supporting a plurality of actuators 114, 116, 118 for controlling various functions of the tool 20 (FIG. 1) of the robot system 10. As illustrated and oriented in FIG. 3, the first actuator 114 is disposed on an outer side surface 112a of the housing 112 in the form of a paddle, the second actuator 116 is disposed on a top surface 112b of the housing 112 in the form of a button, and the third actuator 118 extends from a bottom surface 112c of the housing 112 in the form of a trigger. It should be understood that the first, second, and third actuators 114, 116, 118 can have any suitable configuration (e.g., buttons, knobs, paddles, toggles, slides, triggers, rockers, etc.), and number of and placement of the first, second, and third actuators 114, 116, 118 about the handle assembly 100 may vary. The first actuator 114 includes a finger rest 122 and a strap 124 extending over the finger rest 122 to secure a finger (e.g., the index finger “I”) of the clinician's hand to the first actuator 114 so that the handle assembly 100 does not slide relative to the finger.

With continued reference to FIG. 3, the handle assembly 100 is gripped by a clinician such that the index finger “I” (shown in phantom) of the clinician's hand “H” rests upon the first actuator 114, the palm “L” of the clinician's hand “H” rests on the body and grip portions 110, 120 of the handle assembly 100, and the thumb “T” and the middle finger “M” of the clinician's hand “H” are free to actuate the second and third actuators 116, 118, respectively.

Each handle assembly 100 allows a clinician to manipulate (e.g., clamp, grasp, fire, open, close, rotate, thrust, slice, etc.) the respective tool 20 supported at the end 14 of the arm 12 (FIG. 1). As shown, for example, in FIG. 4, the tool 20 may be a jaw assembly including opposed jaw members 22, 24 extending from a tool shaft 26. The first actuator 114 may be configured to actuate the jaw members 22, 24 of the tool 20 between open and closed configurations. The second and third actuators 116, 118 effect other functions of the tool 20, such as fixing the configuration of the jaw members 22, 24 relative to one another, rotating the jaw members 22, 24 relative to the tool shaft 26, firing a fastener (not shown) from one of the jaw members 22, 24, actuating a knife (not shown) disposed within one of the jaw members 22, 24, activating a source of electrosurgical energy such that electrosurgical energy is delivered to tissue via the jaw members 22, 24, among other functions within the purview of those skilled in the art.

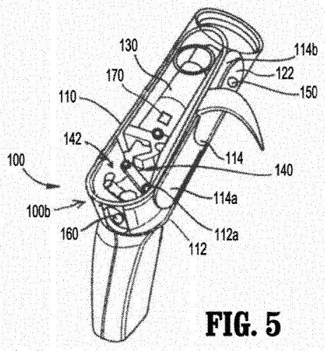

As shown in FIG. 5, a controller 130 is disposed within the body portion 110 of the handle assembly 100 such that actuation of the first, second, and/or third actuator 114, 116, 118 (FIG. 3) actuates the controller 130 which converts mechanical movement of the first, second, and/or third actuators 114, 116, 118 into electrical signals for processing by the processing unit 30 (FIG. 1) which, in turn, sends electrical signals to the robot system 10 (FIG. 1) to actuate a function of the tool 20 (FIG. 1). It should be understood that the robot system 10 may send signals to the processing unit 30 and thus, to the controller 230 to provide feedback to a clinician operating the handle assembly 100.

The first actuator 214 is mechanically coupled to the controller 130 by a linkage assembly 140 including a four-bar linkage 142 and a gear (not shown) rotatable upon movement of the four-bar linkage 142. Actuation of the first actuator 114 causes mechanical movement of a component of the controller 130 which is converted by the controller 130 into an electrical signal. For a detailed discussion of the construction and operation of the four-bar linkage assembly, reference may be made to Int'l Patent Appl. No. PCT/US2017/035583.

The first actuator 114 includes a proximal portion 114a and a distal portion 114b including the finger rest 122. The first actuator 114 has a biased or open position, when no force is applied to the first actuator 114, where the distal portion 114b extends laterally from the outer side surface 112a of the housing 112 of the handle assembly 100 and the proximal portion 114a is flush with, or is disposed within, the outer side surface 112a, as shown in FIG. 5.

In use, when a clinician presses on and applies force to the finger rest 122, the first actuator 114 is moved to an actuated or closed position where the distal portion 114b of the first actuator 114 moves towards the body portion 110 of the handle assembly 100 causing the proximal portion 114a of the first actuator 114 to move laterally away from the body portion 110, resulting in a corresponding movement of the linkage assembly 140. The four-bar linkage 142 act as a crank for rotating the gear (not shown) of the linkage assembly 140 which is meshingly engaged with a gear (not shown) of the controller 130 such that rotation of the gear of the linkage assembly 140 causes a corresponding rotation of the gear of the controller 130. The controller 130 then converts mechanical movement of the gear into electronic signals including digital position and motion information that are transmitted to the processing unit 30 (FIG. 1), as discussed above.

The amount of force applied to the first actuator 114 by a clinician moves the first actuator 114 from the open position to the closed position to affect the position of the jaw members 22, 24 (FIG. 4) with respect to each other. In embodiments, the first actuator 114 is configured such that in the open position, the jaw members 22, 24 are in a fully open position. As a force is applied to the first actuator 114 towards the closed position, the first actuator 114 moves the jaw members 22, 24 towards each other until they reach a fully closed position.

With continued reference to FIG. 5, each of the handle assemblies 100 includes components of a hand detection system. These include a first sensor 150, a second sensor 160, and a third sensor 170. The first sensor 150 is disposed or embedded within the first actuator 114 for sensing the presence of a finger on the first actuator 114, the second sensor 160 is disposed within a proximal end portion 100b of the body portion 110 for sensing the presence of a portion of a hand (e.g., the palm of the hand) about or on the body portion 110, and the third sensor 170 is coupled to or disposed within the controller 130 for measuring the position of the first actuator 114.

In embodiments, the first sensor 150 is a capacitive sensor, the second sensor 160 is an infrared sensor, and the third sensor 170 is an encoder. The first sensor 150 detects changes in a capacitive coupling between the first actuator 114 and the body portion 110 of the handle assembly 100, the second sensor 160 detects changes (e.g., heat or motion) in an area surrounding second sensor 160, and the third sensor 170 detects a position of the first actuator 114. It should be understood that other sensors may be utilized in the handle assemblies 100 for detecting changes in electrical properties (e.g., sensing and/or measuring the presence of objects that are conductive or have a dielectric different from the environment), detecting the proximity of objects, or detecting mechanical motion and generating signals in response to the motion, as is within the purview of those skilled in the art.

The capacitance sensed by the first sensor 150 of the handle assembly 100 changes when a finger is on or in contact with the first actuator 114 and/or with movement of the first actuator 114. The position of the first actuator 114 is correlated with a finger on the finger rest 112 of the first actuator 114 such that the first sensor 150 does not solely detect the presence or absence of a finger thereon. The capacitive coupling changes as the first actuator 114 moves, and is strong or relatively high when the first actuator 114 is in the closed position. Accordingly, as the first actuator 114 approaches or is in the closed position, detecting finger presence on the first actuator 114 becomes difficult.

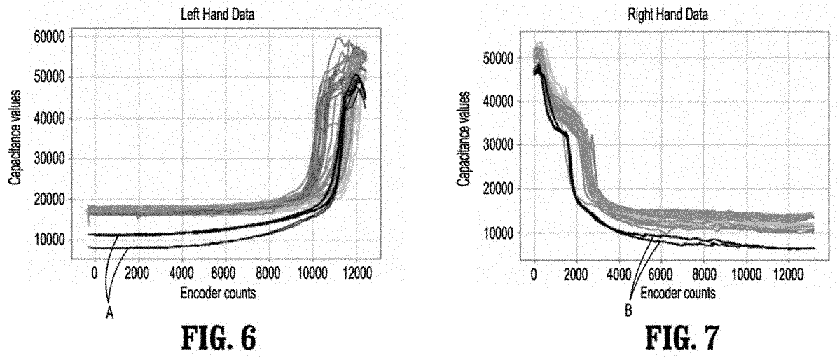

For example, as shown in FIGS. 6 and 7, exemplary curves illustrate capacitance values as a function of encoder counts as the position of the first actuator 114 moves through a full range of motion between the open and closed positions. FIG. 6 shows data corresponding to the handle assembly 100 used in the left hand of a clinician and the FIG. 7 shows data corresponding to the handle assembly 100 used in the right hand of the clinician. The different curves in FIGS. 6 and 7 correspond to different variables during actuation of the first actuator 114 between the open and closed positions, such as wearing and not wearing gloves, different grasps on the handle assembly 100, etc. The two curves labeled “A” in FIG. 6 and “B” in FIG. 7 correspond to no finger being present on the first actuator 114 during the movement between the open and closed positions. As seen in FIGS. 6 and 7, determining whether a finger is present or absent from the first actuator 114 is difficult as the first actuator 114 approaches the closed position and the encoder counts are high.

To detect if the clinician's hand is on the handle assembly 100, the first sensor 150 is utilized to not only sense the presence of a finger thereon, but to also sense the position of the first actuator 114, and data from the first, second, and third sensors 150, 160, 170 are fused or combined through a hand detection algorithm of the hand detection system. The hand detection algorithm is stored as instructions on a computer-readable medium and executed by the processing unit 30 (FIG. 1) and/or in a processing unit (e.g., a microcontroller) of the controller 130. The instructions, when executed by the processing unit 30, cause the hand detection system to determine if a hand is present on the handle assembly 100 and, in turn, to send appropriate signals to the robot system 10 (FIG. 1).

The instructions (e.g., software) of the hand detection system operate during an initialization stage and an operation stage. During the initialization stage, data is recorded that captures the relationship between capacitive value, as sensed by the first sensor 150, and the position of the first actuator 114, as sensed by the third sensor 170, when no hand is present on the handle assembly 100 (e.g., no finger is on the first actuator 114). The recorded data is then processed to construct a lookup table. During the operation stage, the lookup table is used, in conjunction with the first sensor 150, the second sensor 160, and the third sensor 170, to infer hand presence or absence from the handle assembly 100.

During the initialization stage, the response of the first sensor 150 when no hand is present on the handle assembly 100 is measured as a function of the position of the first actuator 114. This measurement occurs during a calibration phase each time the operating console 40 (FIG. 1) initializes, and accounts for the capacitive coupling between the first sensor 150 and the handle assembly 100, for variations between different robot surgical systems and/or components thereof, as well as for other environmental factors. During the calibration phase, the first actuator 114 is slowly swept from the open position to the closed position (e.g., instructions are sent from the hand detection system to a paddle controller of the robotic surgical system) and the capacitive values sensed by the first sensor 150 and the encoder counts generated by the third sensor 170 are recorded simultaneously throughout the motion. This records baseline curves when no finger is present on the first actuator 114 (corresponding to the black curves in FIGS. 6 and 7). The first actuator 114 is swept in both directions (e.g., from the open position to the closed position, and back to the open position) to account for backlash in the first actuator 114.

The data is then processed into a lookup table suitable for real-time use during a surgical procedure in order to infer finger presence on the first actuator 114. Finger presence is inferred if the real-time capacitive value detected by the first sensor 150 exceeds a threshold capacitive value from a calibrated curve generated by the lookup table. The lookup table is designed to enable low-latency access for use in detecting a finger on the first actuator 114.

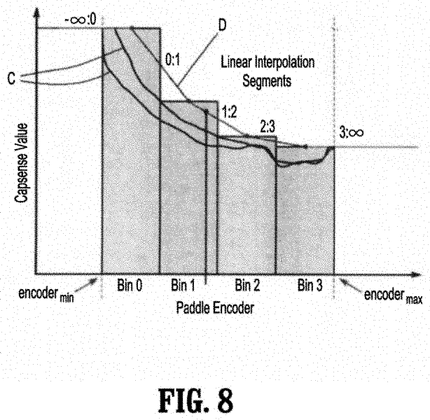

An illustrative lookup table is shown in FIG. 8. The lookup table is parameterized by N, a number of bins, and encoderₘᵢₙ and encoderₘₐₓ, which represent a range of encoder values represented by the lookup table. The width W_{bin} of each bin is:

$${W}_{bin} = \frac{{encoder}_{max} - {encoder}_{min}}{N}$$

Each bin covers a range of encoder values:

binᵢ: [encoderₘᵢₙ+W_{bin}i,encoderₘᵢₙ+W_{bin}(i+1)]

As seen in the lookup table, the bins are shown as rectangles and the baseline curves labeled “C” represent example sensing data (e.g., capacitive values) recorded while sweeping the first actuator 114 during the calibration phase. The calibrated curve labeled “D” denotes the interpolated values that would result from looking up the threshold capacitive value in the lookup table, and are labeled with the bin indicies they fall between.

To construct the lookup table, each point in the recorded data is sorted into the appropriate bin by its encoder count. The threshold capacitive value of the bin is then chosen to be the maximum capacitive value of these points and an error is thrown if there are no points in the bin. The maximum capacitive value is chosen as the threshold capacitive value to decrease the likelihood of falsely detecting a finger on the first actuator 114 when no finger is present.

Once the lookup table is constructed, it can be queried for a capacitive value given an encoder count using linear segments that interpolate between the centers of consecutive bins (see e.g., line “D” in FIG. 8). Given an encoder count, the appropriate pair of consecutive bins is found and an interpolated value is computed. This is a fast constant-time operation by design, as this operation is used in a real-time loop. When querying with an encoder count less than encoderₘᵢₙ or greater than encoderₘₐₓ, the capacitive value of the first or last bin, respectively, is used.

After the initialization stage, the operation stage begins and continues to process while the robotic surgical system 1 remains in use mode. During operation of the handle assembly 100, the lookup table is used, as described above, in conjunction with the first, second, and third sensors 150, 160, 170, to infer hand presence or absence on the handle assembly 100.

Hand presence is inferred using a combination of finger presence on the first sensor 150 (e.g., on the first actuator 114 of the handle assembly 100) and the position of the first actuator 114 as measured by the third sensor 170, and palm presence on the second sensor 160 (e.g., over the proximal end portion 100a of the handle assembly 100).

To detect finger presence, the first sensor 150 is used in conjunction with third sensor 170. If the first actuator 114 is mostly closed (e.g., the encoder count is beyond a certain threshold), then a finger is assumed to be present regardless of the real-time capacitive value sensed by the first sensor 150. This assumption is based, for example, on the fact that the first actuator 114 is biased to spring open without a finger holding it (e.g., due to an applied outward paddle spring torque). Such an assumption allows the real-time capacitive value to be ignored in the challenging regime where differentiating the presence versus absence of a finger is difficult (e.g., when the encoder count is high). Otherwise, if the first actuator 114 is not closed or mostly closed (e.g., the first actuator 114 is moved less than about 70% of the way towards the closed position), a real-time capacitive value is obtained and compared to the threshold capacitive value (corresponding to no finger) via the lookup table. If the real-time capacitive value exceeds this threshold capacitive value, then presence of a finger on the first actuator 114 is inferred. Otherwise, the finger is deduced to be absent from the handle assembly 100.

To detect palm presence, the real-time value (e.g., infrared value) of the second sensor 160 is obtained and checked against a threshold value corresponding to a palm positioned about the handle assembly 100. Palm presence or absence is deduced by checking if the real-time value exceeds the threshold value.

Finally, the finger presence state and the palm presence state are combined to determine a hand presence state (whether or not a hand is present on the handle assembly 100). The hand presence state utilizes a “two in, two out” rule. A positive detection for each of finger presence and palm presence are necessary to transition from a negative to a positive hand presence state. A negative detection for each of finger presence and palm presence are necessary to transition from a positive to a negative hand presence state. Otherwise, no change is made from the standing positive or negative hand presence state. When the hand detection system is in a positive hand presence state, movement of the handle assemblies 100 will cause a corresponding movement in the robot system 10, and when the hand detection system is in a negative hand presence state, the robot system 10 will not move (e.g., be locked) when the handle assemblies 100 are moved.

The hand detection system will also raise exceptions under certain circumstances. For example, the instructions will raise an exception when an insufficient amount of data is used in constructing a lookup table, the data is invalid (e.g., mismatched length of encoder and capacitive sensing values) and/or there is no data corresponding to one or more bins in the lookup table.

The hand detection system may also run tests on the lookup table. Tests may verify that the lookup table correctly interpolates between values based on the data it is provided, that an error is thrown if there is no data within one or more bins of the lookup table, proper operation of the hand detection algorithm, and/or that the hand presence detector behaves properly. For example, a test may generate artificial data resembling actual capacitive sensing data for a hand of a clinician and construct a lookup table for hand detection. Various values of infrared data, capacitive values, and encoder positions are passed in to verify that the “two in, two out” rule is followed (e.g., that both the detection of a finger (via capacitive value and/or encoder count) and detection of a palm (via infrared value) are required to transition to a positive hand presence state, and the detection of no finger and no palm are required to transition to a negative hand presence state), and/or that the system correctly accounts for the case when the first actuator 114 is closed (or mostly closed) and uses the position of the first actuator 114 to detect the presence of a finger.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors of a processing unit, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A robotic surgical system comprising:
a robot system including an arm and a tool coupled to the arm;
a user interface including a handle assembly, the handle assembly including a body portion having a proximal end portion and a distal end portion, the body portion including a first actuator movable between an open position and a closed position;
a hand detection system including a first sensor disposed within the first actuator of the handle assembly for detecting finger presence on the first actuator, a second sensor disposed on the proximal end portion of the handle assembly for detecting palm presence about the proximal end portion, and an encoder disposed within the body portion of the handle assembly for detecting position of the first actuator relative to the body portion; and
a processing unit electrically coupled to the first, second, and third sensors for receiving and processing data from the first, second, and third sensors.

2. The robotic surgical system of claim 1, wherein the first sensor is a capacitive sensor.

3. The robotic surgical system of claim 1, wherein the third sensor is an encoder.

4. The robotic surgical system of claim 1, wherein the second sensor is an infrared sensor.

5. The robotic surgical system of claim 1, wherein, when the hand detection system is in an initialization state, the hand detection system utilizes data from only the first and third sensors, and when the hand detection system is in an operation stage, the hand detection system utilizes data from the first, second, and third sensors.

6. The robotic surgical system of claim 3, wherein, when the hand detection system is in an initialization stage, the first actuator moves through a full range of motion between the open and closed positions, and the first sensor detects a capacitance value at each of a plurality of points through the full range of motion and the third sensor generates an encoder count at each of the plurality of points.

7. The robotic surgical system of claim 6, wherein the hand detection system includes a lookup table including a baseline curve of the capacitance values as a function of the encoder counts and a calibrated curve of threshold capacitance values as a function of the encoder counts.

8. The robotic surgical system of claim 7, wherein, when the hand detection system is in an operation stage, the first sensor detects a real-time capacitance value and the third sensor detects a real-time encoder count, and the real-time capacitance value and the real-time encoder count are compared to the lookup table to identify a positive or negative finger presence state of the handle assembly.

9. The robotic surgical system of claim 8, wherein, when the hand detection system is in an operation stage, the second sensor detects a real-time value which is compared to a threshold value to identify a positive or negative palm presence state of the handle assembly.

10. The robotic surgical system of claim 1, wherein the tool of the robot system is a jaw assembly including opposed jaw members, and when the first actuator is in the open position, the jaw members are in an open configuration, and when the first actuator is in the closed position, the jaw members are in a closed configuration.

11. A method of detecting hand presence on a handle assembly of a robotic surgical system, comprising:
initializing a hand detection system of a robotic surgical system by:
sweeping a first actuator of a handle assembly of the robotic surgical system through a full range of motion from an open position to a closed position;
recording capacitive values obtained from a first sensor disposed within the first actuator of the handle assembly and encoder counts obtained from a third sensor disposed within a body portion of the handle assembly at a plurality of points through the full range of motion; and
constructing a lookup table with the capacitive values as a function of encoder counts at the plurality of points; and
operating the hand detection system by:
comparing a real-time capacitive value of the first sensor and a real-time encoder count of the third sensor against the lookup table to identify a positive or negative finger presence state of the handle assembly.

12. The method of claim 11, wherein operating the hand detection system further includes comparing a real-time value of a second sensor disposed in a proximal end portion of the handle assembly against a threshold value to identify a positive or negative palm presence state of the handle assembly.

13. The method of claim 11, wherein constructing the lookup table includes generating a baseline curve of the capacitance values as a function of the encoder counts and a calibrated curve of threshold capacitance values as a function of the encoder counts.

14. The method of claim 13, wherein comparing the real-time capacitive value of the first sensor and the real-time encoder count of the third sensor against the lookup table includes determining if the real-time capacitive value exceeds the threshold capacitance value.

15. The method of claim 12, further comprising identifying a hand presence detection state where, if positive finger and palm presence states are identified by the hand detection system, a positive hand presence state is identified and movement of the handle assembly results in a corresponding movement of a tool of a robot system, and if negative finger and palm presence states are identified by the hand detection system, a negative hand presence state prevents movement of the tool of the robot system in response to movement of the handle assembly.
